(19) United States
(12) Patent Application Publication
(10) Pub. No.: US 2020/0188273 A1
(43) Pub. Date: Jun. 18, 2020

(54) COMPOSITION AND METHOD FOR TEMPORARILY RESHAPING KERATINOUS FIBRES

(71) Applicant: Henkel AG & Co. KGaA, Duesseldorf (DE)

(72) Inventors: JULIA BIBIANE LANGE, Bad Bramstedt (DE); CYRIELLE MARTINEZ, Hamburg (DE); DIANE METTEN, Hamburg (DE)

(73) Assignee: Henkel AG & Co. KGaA, Duesseldorf (DE)

(21) Appl. No.: 16/717,515

(22) Filed: Dec. 17, 2019

(30) Foreign Application Priority Data
Dec. 18, 2018 (DE) 10 2018 222 043.3

(51) Int. Cl. A61K 8/73; A61K 8/81; A61Q 5/06; A61K 8/41; A61K 8/36

(52) U.S. Cl. CPC A61K 8/737; A61K 8/8141; A61K 8/8176; A61K 8/8182; A61K 2800/594; A61Q 5/06; A61K 8/41; A61K 8/36; A61K 2800/5426; A61K 8/8135

(57) ABSTRACT

A cosmetic composition for temporary reshaping keratinous fibers, having:
a) a cationically modified guar derivative having a weight-average molecular weight in the range of from about 5,000 to about 200,000 and a cationic degree of substitution in the range of from about 0.1 to about 2 and
b) at least one polyimide, and
   their use and a method of using such a composition.

### CROSS-REFERENCE TO RELATED APPLICATION

This application claims priority to German Patent Application No. 10 2018 222 043.3, filed Dec. 18, 2018, which is incorporated herein by reference in its entirety.

### TECHNICAL FIELD

The present disclosure relates to a cosmetic composition based on two selected polymers for hair setting or for temporary reshaping of keratinous fibers, in particular human hair, and methods of using this composition.

### BACKGROUND

The temporary design of hairstyles for a longer period of time of up to several days usually requires the application of firming active substances. Hair treatment agents which provide a temporary shaping of the hair therefore play an important role. Corresponding compositions for temporary reshaping usually contain synthetic polymers and/or waxes as a firming active substance. Compositions for supporting the temporary reshaping of keratinous fibers can be formulated, for example, as a hair spray, hair wax, hair gel or hair foam.

The most important property of an agent for the temporary reshaping of hair, hereinafter also referred to as a styling agent, is to give the treated hair the strongest possible hold in the newly modeled shape, that is, a shape impressed on the hair. One also speaks of strong hairstyle hold or the high degree of hold of the styling agent. The hairstyle hold is substantially determined by the type and amount of firming active substance used, wherein, however, further constituents of the styling agent can also have an influence.

Styling agents must meet a whole series of other requirements in addition to a high degree of hold. These can be roughly subdivided into properties on the hair, properties of the particular formulation, for example, properties of the foam, the gel or the sprayed aerosol, and properties relating to the handling of the styling agent, wherein the properties on the hair are accorded particular importance. Particularly noteworthy are moisture resistance, low tack and a balanced conditioning effect. Furthermore, a styling agent should be universally applicable for all hair types and mild to hair and skin.

The hairstyle in general and curl retention for wavy hair are special requirements for styling agents. So-called curl retention is a measure of degree of curl hold. Usually, curl retention becomes worse when the treated hair is in a humid environment, since the tendency of the hair to absorb moisture, that is, water, reduces curl hold.

A variety of synthetic polymers which are used in styling agents have already been developed as firming active substances to meet the different requirements. The polymers can be subdivided into cationic, anionic, nonionic and amphoteric firming polymers.

The international patent application WO 2012/075274 A1 describes an exemplary styling agent having a polyimide.

### BRIEF SUMMARY

Cosmetic compositions and methods for their use are provided. In an exemplary embodiment, a cosmetic composition includes a modified guar derivative a) having a weight-average molecular weight in the range of from about 5,000 to about 200,000. The modified guar derivative a) has a cationic degree of substitution in the range of from about 0.1 to about 2. The cosmetic composition further includes a polyimide b).

A method of using a cosmetic composition is provided in another embodiment. The method includes temporarily fixing keratinic fibers into a shape, and applying the cosmetic composition to the keratinic fibers. The cosmetic composition includes a modified guar derivative a) and a polyimide b). The modified guar derivative a) has a weight-average molecular weight in the range of from about 5,000 to about 200,000, and a cationic degree of substitution in the range of from about 0.1 to about 2.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It was an object of the present disclosure to provide further suitable polymer combinations which are distinguished by good film-forming and/or firming properties, having a very high degree of hold, without having to forgo flexibility and good moisture resistance, in particular perspiration and water resistance. It is a particular object of the present disclosure to provide such styling agents which provide both high long-term hold and high curl retention in a humid environment.

This was achieved as contemplated herein by a combination of two selected polymers.

The present disclosure provides:

1. A cosmetic composition for temporary reshaping keratinous fibers, containing:
a) at least one cationically modified guar derivative having a weight-average molecular weight in the range of from about 5,000 to about 200,000 and a cationic degree of substitution in the range of from about 0.1 to about 2 and
a) at least one polyimide.

2. The cosmetic composition as contemplated herein, wherein the weight proportion of the cationically modified guar derivative a) is from about 0.1 to about 10% by weight, preferably from about 0.15 to about 5% by weight and in particular from about 0.2 to about 2.5% by weight, based on a total weight of the cosmetic composition.

3. The cosmetic composition as contemplated herein, wherein the cationically modified guar derivative a) has a weight-average molecular weight in the range of from about 20,000 to about 150,000, more preferably in the range of from about 35,000 to about 100,000, and most preferably in the range of from about 50,000 to about 70,000.

4. The cosmetic composition as contemplated herein, wherein the cationically modified guar derivative a) has a cationic degree of substitution in the range of from about 0.2 to about 1.

5. The cosmetic composition as contemplated herein, wherein the cationically modified guar derivative a) is selected from the group of compounds having the INCI name Guar Hydroxypropyltrimonium Chloride.

6. The cosmetic composition as contemplated herein, wherein the weight proportion of the polyimide b) is from about 0.1 to about 10% by weight, preferably from about 0.15 to about 5% by weight and in particular from about 0.2 to about 2.5% by weight, based on the total weight of the composition.

7. The cosmetic composition as contemplated herein, wherein the polyimide has at least one structural unit (A1), at least one structural unit (A2), at least one structural unit (A3) and at least one structural unit (A4),

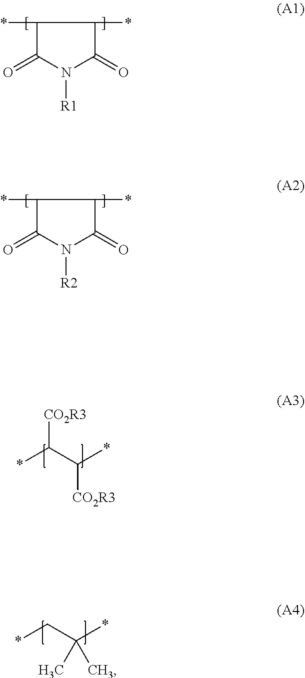

wherein
R1 stands for an optionally heterofunctionalized alkyl radical;
R2 stands for an optionally heterofunctionalized alkyl radical other than R1; and
R3, independently of one another, stands for an optionally heterofunctionalized alkyl radical other than R1 and R2.

8. The cosmetic composition as contemplated herein, wherein the polyimide b) is selected from the group of compounds having the INCI name isobutylene/dimethylaminopropyl maleimide/ethoxylated maleimide/maleic acid copolymer (Polyimide-1).

9. The cosmetic composition as contemplated herein, wherein the weight ratio of the cationically modified guar derivative a) to polyimide b) is from about 10:1 to about 1:10, preferably from about 5:1 to about 1:5 and more preferably from about 3:1 to about 1:3 and particularly preferred from about 1.1:1 to about 1:1.1.

10. The cosmetic composition as contemplated herein, further contains
c) polyvinylpyrrolidone and/or vinylpyrrolidone/vinyl acetate copolymer, preferably polyvinylpyrrolidone.

11. The cosmetic composition as contemplated herein, wherein the weight proportion of the polyvinylpyrrolidone and/or vinylpyrrolidone/vinyl acetate copolymer c) is from about 0.1 to about 10% by weight, preferably from about 2 to about 8.5% by weight and in particular from about 3 to about 7% by weight in the total weight of the cosmetic composition.

12. The cosmetic composition as contemplated herein, wherein the composition, based on its total weight, contains from about 0.01 to about 5% by weight, preferably from about 0.02 to about 4% by weight and in particular from about 0.05 to about 2% by weight of an organic acid or its salt, preferably lactic acid or its salt.

13. The cosmetic composition as contemplated herein, wherein the cosmetic composition, based on its total weight, contains from about 0.01 to about 5% by weight, more preferably from about 0.01 to about 2% by weight and particularly preferably from about 0.02 to about 1.5% by weight of an alkanolamine or its neutralized form, in particular 2-amino-2-methylpropanol or its neutralized form.

14. The cosmetic composition as contemplated herein, wherein the composition, based on its total weight, contains at least about 20% by weight, preferably at least about 40% by weight and in particular at least about 65% by weight of water.

15. The cosmetic composition as contemplated herein, exemplified in that the composition is present as a hair gel, a hair spray, a hair foam, hair cream, or a hair wax.

16. A use of a cosmetic composition as contemplated herein for temporarily reshaping keratinous fibers, in particular human hair.

17. A use of a cosmetic composition as contemplated herein for improving the moisture resistance of temporarily reshaped keratinous fibers.

18. A use of a cosmetic composition as contemplated herein for improving curl retention of temporarily reshaped keratinous fibers in a humid environment.

19. A method for temporarily reshaping keratinous fibers, in particular human hair, in which the keratinous fibers are acted upon by a cosmetic composition as contemplated herein and temporarily fixed in their shape.

Cationic guar derivatives are used in hair care products to care for the hair, for example, to impart improved combability to the hair. Cationic guar derivatives can impart conditioning effects to the skin in skin care products. In detergent and softener formulations, cationic guar derivatives impart conditioning, softening, abrasion-resistance and anti-static properties to fabrics treated therewith.

All the more surprisingly, it has been found that a high hold can be achieved by styling products using selected cationic guar derivatives in combination with a firming/film-forming styling polymer, polyimide b), which is already used in styling products. Other commonly required properties of styling products such as long-term hold, stiffness and low tack remained intact. Such a good combination of properties was not expected even with knowledge of the individual components and was surprising. It was furthermore found that the combination of the two components resulted in a strongly super-additive, that is, synergistic, effect with regard to moisture resistance, in particular with regard to the curl retention in a humid environment, which manifested itself in the HHCR test (High Humidity Curl Retention Test).

The term keratinous fibers as contemplated herein comprises furs, wool and feathers, but in particular human hair. Human hair can include head and/or beard hair.

The essential constituents of the cosmetic composition are the cationic guar derivative a) and the polyimide b).

The cosmetic compositions are distinguished from alternative cosmetic compositions in particular by an improved curl retention in a humid environment. For the cosmetic properties of the compositions, a weight ratio of the cationically modified guar derivative a) and the polyimide b) in the cosmetic composition of from about 10:1 to about 1:10, preferably from about 5:1 to about 1:5 and in particular from about 3:1 to about 1:3 has proved advantageous. It is extremely advantageous when the weight ratio of the cationically modified guar derivative a) to polyimide b) is about 1:1, in particular in the range from about 1.1:1 to about 1:1.1.

The cosmetic compositions contain a cationic guar derivative a) as a first essential component.

In the context of this application, the term “guar derivatives” is understood to mean (bio)chemically and/or physically modified guar. Guar gum is a polysaccharide of galactose and mannose, which has a linear backbone of β-1,4-linked mannose units. Galactose units are bound via β-1,6-glycosidic bonds on every second mannose unit of this backbone. The chemical modification of these guar gums can be carried out, for example, by esterification or etherification of the hydroxyl groups of the polysaccharide or by reaction with bases, acids or oxidizing agents. A biochemical modification of this guar can be carried out, for example, by reaction with hydrolytic enzymes, bacteria or fungi. A physical modification is possible, for example, using heat, radiation and comminution, for example, by employing a high-speed stirrer.

“Cationically modified guar derivatives” are understood to mean guar gums whose hydroxy groups have been esterified or etherified with a compound which has at least one cationic group. This cationic group can be either permanently cationic or temporarily cationic. As contemplated herein, “permanently cationic” refers to compounds which have a cationic group, regardless of the pH value of the cosmetic composition. These include in particular compounds having quaternary nitrogen atoms, such as quaternary ammonium groups. On the other hand, “temporary cationic” refers to those compounds which have a cationic group only at certain pH values, in particular pH values in the acidic range. Examples of temporary cationic groups are amine groups.

The cationic functionality can be added by a variety of methods. For example, the starting material can be reacted for sufficient time and at sufficient temperature with a tertiary amine compound or a quaternary ammonium compound containing groups capable of reacting with reactive groups of the guar, in particular the hydroxy groups.

Suitable compounds for introducing the cationic functionality comprise, for example, 2-dialkylaminoethyl chloride and quaternary ammonium compounds such as 3-chloro-2-hydroxypropyltrimethylammonium chloride and 2,3-epoxypropyltrimethylammonium chloride. Further examples are glycidyl trialkyl ammonium salts and 3-halo-2-hydroxypropyl trialkyl ammonium salts such as glycidyl trimethyl ammonium chloride, glycidyl triethyl ammonium chloride, glycidyl tripropyl ammonium chloride, glycidyl ethyl dimethyl ammonium chloride, glycidyl diethyl methyl ammonium chloride and the corresponding bromides and iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride and their corresponding bromides and iodides; and quaternary ammonium compounds such as halides of compounds containing an imidazoline ring.

It is preferred that the cationically modified guar derivative a) comprises at least one structural unit of the formula (I),

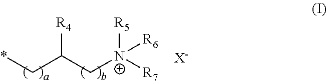

wherein
R₄ stands for hydrogen, a C₁₋₄ alkyl group or a hydroxyl group,
R₅, R₆ and R₇ each independently of one another, stand for a C₁₋₈ alkyl group, a and b independently of one another, each stand for an integer from 1 to 3,
X− stands for a physiologically compatible anion.

Examples of C₁₋₄ alkyl groups are methyl, ethyl, propyl, isopropyl, butyl, sec-butyl, isobutyl or tert-butyl groups.

Examples of C₁₋₈ alkyl groups are methyl, ethyl, propyl, isopropyl, butyl, sec-butyl, isobutyl, tert-butyl, pentyl and hexyl, heptyl and octyl groups.

In the structural unit of the formula (I), the radical R₄ preferably stands for a hydroxyl group and a and b, in each case independently of one another, stand for the integer 1.

Moreover, it is preferred that in the structural unit of the formula (I), the radicals R₅ to R₇, each independently of one another, stands for a C₁₋₆ alkyl group, preferably a C₁₋₄ alkyl group, more preferably a C₁₋₃ alkyl group, in particular a C₁ alkyl group, and X⁻ stands for a halide ion, in particular chloride.

Other derivatizations of the cationic guar derivative with nonionic substituents, that is, hydroxyalkyl, wherein the alkyl constitutes a straight or branched chain hydrocarbon radical of 1 to 6 carbon atoms (for example, hydroxyethyl, hydroxypropyl, hydroxybutyl), or anionic substituents such as carboxymethyl groups, are optional. These optional substituents can be added to the cationic guar derivative by reaction with reagents such as (1) alkylene oxides (for example, ethylene oxide, propylene oxide, butylene oxide) to obtain hydroxyethyl groups, hydroxypropyl groups or hydroxybutyl groups or with (2) chloromethylacetic acid to obtain a carboxymethyl group. However, it is highly preferred that the cationic guar derivative a) has no further substituents, whether nonionic or anionic in nature.

The cationically modified guar derivative a) has a weight-average molecular weight in the range of from about 5,000 to about 200,000. It is preferred that the cationically modified guar derivative a) has a weight-average molecular weight in the range of from about 20,000 to about 150,000, more preferably in the range of from about 35,000 to about 100,000, and most preferably in the range of from about 50,000 to about 70,000.

The weight-average molecular weight can be determined, for example, by gel permeation chromatography using a polystyrene standard.

The cationically modified guar derivative a) has a cationic degree of substitution (DS) in the range from about 0.1 to about 2. It is preferable that the degree of cationic substitution is in the range of from about 0.2 to about 1.

It is particularly advantageous when the cationically modified guar derivative a) has a degree of substitution (DS) by the structural unit of the formula (I) of from about 0.1 to about 2, in particular from about 0.2 to about 1.

The degree of substitution (DS) describes the average number of cationic structural units, in particular of cationic structural units of the formula (I), which are bonded per monomer of the polysaccharide, that is, per anhydromannose and anhydrogalactose. Since each monomer of the polysaccharide has on average 3 free OH groups, the DS can assume values between 0 and 3. Thus, for example, a DS value of 1 means that on average one cationic structural unit is bound per monomer of the polysaccharide, so that each monomer furthermore has 2 free OH groups. The degree of substitution (DS) can be determined, for example, by employing ¹H NMR spectroscopy or titration.

It is highly preferred that the cationically modified guar derivative comprises a cationically modified guar derivative having the INCI name “Guar Hydroxypropyltrimonium Chloride” which has a weight-average molecular weight in the range of from about 5,000 to about 200,000 and a cationic degree of substitution in the range of from about 0.1 to about 2. Such a cationically modified guar derivative is commercially available, for example, under the name “N-Hance CCG 45 Cationic Guar” from Ashland Specialty Chemical.

The weight proportion of the cationic guar derivative a) in the total weight of the composition is preferably from about 0.1 to about 10% by weight, more preferably from about 0.15 to about 5% by weight and in particular from about 0.2 to about 2.5% by weight.

The cosmetic compositions contain at least one polyimide b) as a second essential constituent.

In a preferred embodiment, the polyimide has at least one structural unit (A1), at least one structural unit (A2), at least one structural unit (A3) and at least one structural unit (A4),

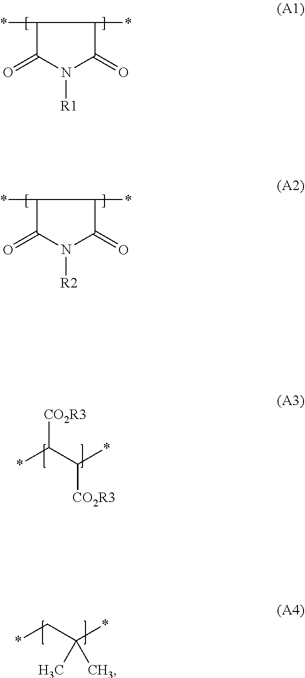

wherein
R1 stands for an optionally heterofunctionalized alkyl radical;
R2 stands for an optionally heterofunctionalized alkyl radical other than R1; and
R3, independently of one another, stands for an optionally heterofunctionalized alkyl radical other than R1 and R2.

With regard to the cosmetic properties of the cosmetic composition, it has proved to be advantageous when
radical R1 in formula (A1) stands for a radical —CH(CH₃)CH₂—(OCH(CH₃)CH₂)ₓ(O[CH₂]₂)_{y}OCH₃, in which x and y, independently of one another, have a value between 1 and about 100 and/or
radical R2 in the structural unit (A2) stands for a radical containing an amino group, preferably for a radical having a tertiary amine, and/or
at least one of the radicals R3 of the structural unit (A3) stands for an alkyl radical, preferably a (C1 to C4) alkyl radical.

Particularly preferred polyimides include at least about 70% by weight, preferably at least about 80% by weight, preferably at least 90% by weight and in particular at least about 95% by weight of the structural units (A1), (A2), (A3) and (A4). Further preferred polyimides are formed entirely of the structural units (A1), (A2), (A3) and (A4).

Some preferred polyimides are listed below. These polyimides include at least about 70% by weight, preferably at least about 80% by weight, preferably at least about 90% by weight and in particular at least about 95% by weight, most preferably entirely of the structural units (A1), (A2), (A3) and (A4):

A-I) polyimides having at least one structural unit (A1), at least one structural unit (A2), at least one structural unit (A3) and at least one structural unit (A4),

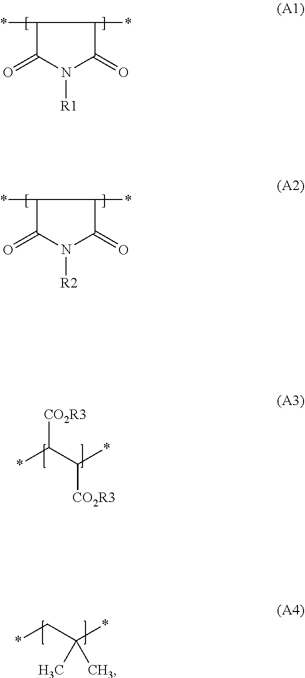

wherein
R1 in formula (A1) stands for a radical —CH(CH₃)CH₂—(OCH(CH₃)CH₂)ₓ(O[CH₂]₂)_{y}OCH₃, in which x and y, independently of one another, have a value between 1 and about 100 and/or
R2 in formula (A2) stands for a radical —(CH₂)₃—N(CH₃)₂;
R3, independently of one another, stands for an optionally heterofunctionalized alkyl radical other than R1 and R2.

A-II) polyimides having at least one structural unit (A1), at least one structural unit (A2), at least one structural unit (A3) and at least one structural unit (A4),

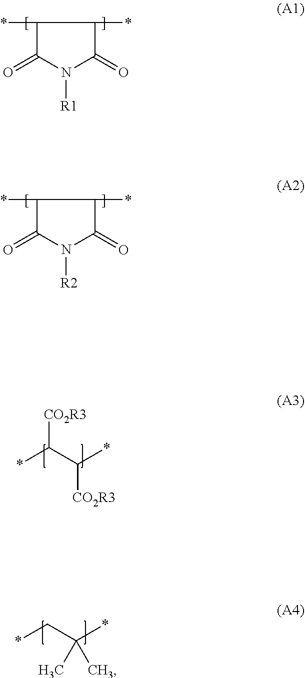

wherein
R1 in formula (A1) stands for a radical —CH(CH₃)CH₂—(OCH(CH₃)CH₂)ₓ(O[CH₂]₂)_{y}OCH₃, in which x and y, independently of one another, have a value between 1 and about 100 and/or
R2 stands for an optionally heterofunctionalized alkyl radical other than R1; and
a radical R3 in the formula (A3) stands for —CH₂CH₃ or —CH₂CH₂CH₃, preferably for —CH₂CH₃ and a radical R3 in the formula (A3) is H.

A-III) polyimides having at least one structural unit (A1), at least one structural unit (A2), at least one structural unit (A3) and at least one structural unit (A4),

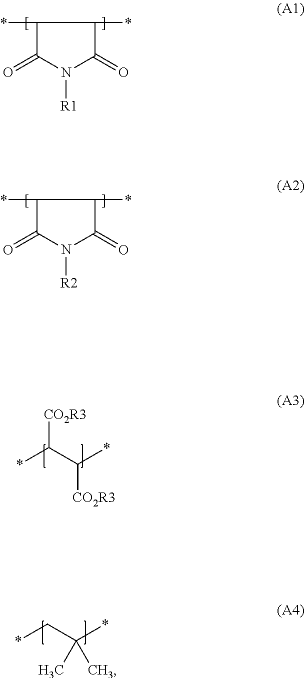

wherein
R1 stands for an optionally heterofunctionalized alkyl radical;
R2 in formula (A2) stands for a radical —(CH₂)₃—N(CH₃)₂; and
a radical R3 in the formula (A3) stands for —CH₂CH₃ or —CH₂CH₂CH₃, preferably for —CH₂CH₃ and a radical R3 in the formula (A3) is H.

Very particularly preferred polyimides are exemplified in that
the radical R1 in formula (A1) stands for a radical —CH(CH₃)CH₂—(OCH(CH₃)CH₂)ₓ(O[CH₂]₂)_{y}OCH₃, in which x and y, independently of one another, have a value between 1 and about 100;
the radical R2 in formula (A2) stands for a radical —(CH₂)₃—N(CH₃)₂;
a radical R3 stands for —CH₂CH₃ and a radical R3 in formula (A3) stands for H.

Suitable polyimides are available, for example, from Ashland under the trade names Aquaflex XL 30 (INCI: isobutylene/dimethylaminopropyl maleimide/ethoxylated maleimide/maleic acid copolymer or Polyimide-1).

The weight proportion of the polyimides b) in the total weight of the cosmetic agent is preferably from about 0.1 to about 10% by weight, more preferably from about 0.15 to about 5% by weight and in particular from about 0.2 to about 2.5% by weight.

It can be preferred for the cosmetic composition to contain one or more further polymer(s) other than polymers a) and b), for example, to aid the thickening agents or gelation or film formation. Examples are cationic, anionic, nonionic or amphoteric polymers.

Examples are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacryl amide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polyquaternium-68, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

The further component acting as gelling agent is preferably a homopolyacrylic acid (INCI: Carbomer), which is commercially available, for example, under the name Carbopol® in different designs. Carbomer is preferably present in a proportion of from about 0.02 to about 3% by weight, preferably from about 0.05 to about 1.5% by weight, and more preferably from about 0.2 to about 0.8% by weight, based on the total weight of the cosmetic composition.

To further increase their cosmetic effect, preferred compositions, in addition to the polymers a) and b) and an optionally added thickening agent or gelling agent further comprise a film-forming polymer c) other than these ingredients, in particular an anionic or nonionic polymer c).

Examples of nonionic polymers are:
vinylpyrrolidone/vinyl ester copolymers, such as those sold under the name Luviskol (BASF). Luviskol VA 64 and Luviskol VA 73, in each case vinylpyrrolidone/vinyl acetate copolymers, are preferred nonionic polymers.
cellulose ethers, such as hydroxypropylcellulose, hydroxyethylcellulose and methylhydroxypropylcellulose, as sold, for example, under the names Culminal and Benecel (AQUALON).
shellac.
polyvinylpyrrolidones, as they are sold, for example, under the name Luviskol (BASF).
siloxanes. These siloxanes can be both water-soluble and water-insoluble. Both volatile and nonvolatile siloxanes are suitable, wherein nonvolatile siloxanes are understood to mean those compounds whose boiling point is above about 200° C. under normal pressure. Preferred siloxanes are polydialkylsiloxanes, such as, for example, polydimethylsiloxane, polyalkylarylsiloxanes, such as, polyphenylmethylsiloxane, ethoxylated polydialkylsiloxanes and polydialkylsiloxanes which contain amine and/or hydroxyl groups.
glycosidically substituted silicones.

Due to their cosmetic effect in combination with the copolymers a) and b), preferably used film-forming polymers are the polyvinylpyrrolidones (INCI name: PVP) and the vinylpyrrolidone/vinyl acetate copolymers (INCI name VP/VA copolymer). The holding properties, but also the application properties of the cosmetic compositions, are noticeably influenced by adding film-forming polymers, in particular the aforementioned polyvinylpyrrolidones and vinylpyrrolidone/vinyl acetate copolymers. The weight proportion of these polymers is preferably limited to amounts between about 1.0 and about 10% by weight. Preferred cosmetic compositions, based on their total weight, further contain from about 1 to about 10% by weight of polyvinylpyrrolidone and/or vinylpyrrolidone/vinyl acetate copolymer, preferably polyvinylpyrrolidone. Particularly preferred cosmetic compositions have a weight proportion of the polyvinylpyrrolidone and/or vinylpyrrolidone/vinyl acetate copolymer c) of from about 2 to about 8.5% by weight, preferably from about 3 to about 7% by weight based on the total weight of the cosmetic composition.

The cosmetic composition can contain further conventional ingredients of styling products. In particular, additional care substances can be mentioned as further suitable auxiliaries and additives.

The composition, for example, can contain at least one protein hydrolyzate and/or one of its derivatives as a care substance. Protein hydrolyzates are product mixtures which can be obtained by acid, alkaline or enzymatically catalyzed degradation of proteins. The term protein hydrolyzates is also understood to mean total hydrolyzates and also individual amino acids and their derivatives and mixtures of different amino acids.

The composition can further contain at least one vitamin, one provitamin, one vitamin precursor and/or one of their derivatives as a care substance. In this case, those vitamins, provitamins and vitamin precursors are preferred, which are usually assigned to the groups A, B, C, E, F and H.

As with the addition of glycerol and/or propylene glycol, the addition of panthenol increases the flexibility of the polymer film formed upon application of the composition.

The compositions can further contain at least one plant extract, but also mono- or oligosaccharides and/or lipids as a care substance.

Furthermore, oil bodies are suitable as a care substance. The natural and synthetic cosmetic oil bodies include, for example, vegetable oils, liquid paraffin oils, isoparaffin oils and synthetic hydrocarbons and di-n-alkyl ethers having a total of from about 12 to about 36 carbon atoms, in particular from about 12 to about 24 carbon atoms. Preferred cosmetic compositions contain at least one oil body, preferably at least one oil body from the group of silicone oils. The group of silicone oils includes in particular the dimethicones, to which the cyclomethicones are also to be counted, the amino-functional silicones and the dimethiconols. The dimethicones can be both linear and branched and cyclic or cyclic and branched. Suitable silicone oils or silicone gums are, in particular, dialkyl and alkylaryl siloxanes, such as dimethylpolysiloxane and methylphenylpolysiloxane, and also their alkoxylated, quaternized or else anionic derivatives. Preference is given to cyclic and linear polydialkylsiloxanes, and their alkoxylated and/or aminated derivatives, such as dihydroxypolydimethylsiloxanes and polyphenylalkylsiloxanes.

Ester oils, that is, esters of C6-C30 fatty acids with C2-C30 fatty alcohols, preferably monoesters of fatty acids with alcohols having from 2 to about 24 carbon atoms such as isopropyl myristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, cocofatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are further preferred care oil bodies.

Further suitable care substances are dicarboxylic acid esters, symmetrical, asymmetrical or cyclic esters of carbonic acid with fatty alcohols, triflic acid esters of saturated and/or unsaturated linear and/or branched fatty acids with glycerol or fatty acid partial glycerides, which are understood to mean monoglycerides, diglycerides and technical mixtures thereof.

Furthermore, emulsifiers or surface-active substances are preferably present in the composition. Preference is given to PEG derivatives of hydrogenated Castor oil, which, for example, are available under the name PEG Hydrogenated Castor Oil, for example, PEG-30 Hydrogenated Castor Oil, PEG-35 Hydrogenated Castor Oil, PEG-36 Hydrogenated Castor Oil or PEG-40 Hydrogenated Castor Oil. The use of PEG-40 Hydrogenated Castor Oil is preferred. These are preferably present in an amount of from about 0.05 to about 1.5% by weight, more preferably from about 0.1 to about 1% by weight, also preferably from about 0.2 to about 0.8% by weight or from about 0.3 to about 0.6% by weight. In particular, the washability of the cosmetic compositions, in addition to the processibility, is improved by adding the surface-active substances, in particular the aforementioned PEG derivatives of hydrogenated Castor oil.

The cosmetic compositions contain the ingredients or active substance in a cosmetically acceptable carrier.

Preferred cosmetically acceptable carriers are aqueous, alcoholic or aqueous-alcoholic media having preferably at least about 10% by weight of water, calculated on the total weight of the composition. The cosmetic carrier particularly preferably contains water, in particular in an amount such that the cosmetic composition contains at least about 20% by weight, in particular at least about 40% by weight, most preferably at least about 65% by weight of water, based on its total weight. Very particularly preferred cosmetic compositions have, based on their total weight, a water proportion of from about 50 to about 95% by weight, preferably from about 60 to about 90% by weight and in particular from about 65 to about 85% by weight.

It is possible in particular to include the lower alcohols having 1 to 4 carbon atoms usually used for cosmetic purposes, such as, for example, ethanol and isopropanol, as alcohols.

Examples of water-soluble solvents as a co-solvent are glycerol and/or ethylene glycol and/or 1,2-propylene glycol in an amount of from about 0 to about 30% by weight based on the total composition.

It can be preferred that the composition contains an organic acid or its salt. The organic acid is preferably selected from the group including maleic acid, lactic acid, acetic acid, propionic acid, citric acid, tartaric acid, succinic acid, oxalic acid, gluconic acid, malic acid, amino acids and mixtures thereof. Most preferably, the organic acid comprises lactic acid.

The amount of organic acid or its salt, preferably of lactic acid or its salt, is preferably from about 0.01 to about 5% by weight, more preferably from about 0.02 to about 4% by weight, and particularly preferably from about 0.05 to about 2% by weight in the total weight of the cosmetic composition.

The cationically modified guar derivative is preferably used in the form of an acidic, aqueous solution. An organic acid is preferably used for acidifying the aqueous solution.

It can be preferred that the cosmetic composition further contains an alkanolamine The alkanolamines which can be used as alkalizing agents are preferably selected from primary amines having a C₂-C₆ alkyl basic body which carries at least one hydroxyl group. Further suitable alkanolamines are selected from the group formed of 2-aminoethane-1-ol (monoethanolamine), tris(2-hydroxyethyl) amine (triethanolamine), 3-aminopropan-1-ol, 4-aminobutan-1-ol, 5-aminopentan-1-ol, 1-aminopropane-2 ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-amino-2-methylpropane-1,3-diol. Very particularly preferred alkanolamines are selected from the group 2-aminoethane-1-ol, 2-amino-2-methylpropan-1-ol and 2-amino-2-methyl-propane-1,3-diol. 2-amino-2-methylpropanol has proven to be particularly suitable. Preferably, the weight proportions of the amino alcohol or its neutralized form, preferably of 2-amino-2-methylpropanol, are from about 0.01 to about 5% by weight, more preferably from about 0.01 to about 2% by weight and in particular preferably from about 0.02 to about 1.5% by weight, based on the total weight of the cosmetic composition.

The composition of some preferred cosmetic compositions can be found in the following table (specifications in % by weight based on the total weight of the cosmetic composition, unless stated otherwise).

| | Formula | Formula | Formula | Formula | Formula |
|---|---|---|---|---|---|
| | 1 | 2 | 3 | 4 | 5 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Misc | ad 100 | ad 100 | ad 100 | ad 100 | ad 100 |
| | Formula | Formula | Formula | Formula | Formula |
| | 1a | 2a | 3a | 4a | 5a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Misc | ad 100 | ad 100 | ad 100 | ad 100 | ad 100 |
| | Formula | Formula | Formula | Formula | Formula |
| | 1b | 2b | 3b | 4b | 5b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Misc | ad 100 | ad 100 | ad 100 | ad 100 | ad 100 |
| | Formula | Formula | Formula | Formula | Formula |
| | 6 | 7 | 8 | 9 | 10 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Misc | ad 100 | ad 100 | ad 100 | ad 100 | ad 100 |
| | Formula | Formula | Formula | Formula | Formula |
| | 6a | 7a | 8a | 9a | 10a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Misc | ad 100 | ad 100 | ad 100 | ad 100 | ad 100 |
| | Formula | Formula | Formula | Formula | Formula |
| | 6b | 7b | 8b | 9b | 10b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Polyvinylpyrrolidone | 1 to 10 | 2 to 8.5 | 2 to 8.5 | 3 to 7 | 3 to 7 |
| Misc | ad 100 | ad 100 | ad 100 | ad 100 | ad 100 |
| | Formula | Formula | Formula | Formula | Formula |
| | 11 | 12 | 13 | 14 | 15 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Misc | ad 100 | ad 100 | ad 100 | ad 100 | ad 100 |
| | Formula | Formula | Formula | Formula | Formula |
| | 11a | 12a | 13a | 14a | 15a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Misc | ad 100 | ad 100 | ad 100 | ad 100 | ad 100 |
| | Formula | Formula | Formula | Formula | Formula |
| | 11b | 12b | 13b | 14b | 15b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Vinylpyrrolidone/vinyl | 1 to 10 | 2 to 8.5 | 2 to 8.5 | 3 to 7 | 3 to 7 |
| acetate copolymer | | | | | |
| Misc | ad 100 | ad 100 | ad 100 | ad 100 | ad 100 |
| | Formula | Formula | Formula | Formula | Formula |
| | 16 | 17 | 18 | 19 | 20 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |
| | Formula | Formula | Formula | Formula | Formula |
| | 16a | 17a | 18a | 19a | 20a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |
| | Formula | Formula | Formula | Formula | Formula |
| | 16b | 17b | 18b | 19b | 20b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |
| | Formula | Formula | Formula | Formula | Formula |
| | 16c | 17c | 18c | 19c | 20c |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |
| | Formula | Formula | Formula | Formula | Formula |
| | 16d | 17d | 18d | 19d | 20d |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |
| | Formula | Formula | Formula | Formula | Formula |
| | 21 | 22 | 23 | 24 | 25 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | ad 100 | ad 100 | ad 100 | ad 100 | ad 100 |
| | Formula | Formula | Formula | Formula | Formula |
| | 21a | 22a | 23a | 24a | 25a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | ad 100 | ad 100 | ad 100 | ad 100 | ad 100 |
| | Formula | Formula | Formula | Formula | Formula |
| | 21b | 22b | 23b | 24b | 25b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XE-30 | 0.1 to 10 | 0.15 to 5 | 0.15 to 5 | 0.2 to 2.5 | 0.2 to 2.5 |
| (specification as | | | | | |
| solid content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Misc | ad 100 | ad 100 | ad 100 | ad 100 | ad 100 |
| | Formula | Formula | Formula | Formula | Formula |
| | 26 | 27 | 28 | 29 | 30 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 26a | 27a | 28a | 29a | 30a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 26b | 27b | 28b | 29b | 30b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 31 | 32 | 33 | 34 | 35 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 31a | 32a | 33a | 34a | 35a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 31b | 32b | 33b | 34b | 35b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Polyvinylpyrrolidone | 1 to 10 | 2 to 8.5 | 2 to 8.5 | 3 to 7 | 3 to 7 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 36 | 37 | 38 | 39 | 40 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 36a | 37a | 38a | 39a | 40a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 36b | 37b | 38b | 39b | 40b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Vinylpyrrolidone/vinyl | 1 to 10 | 2 to 8.5 | 2 to 8.5 | 3 to 7 | 3 to 7 |
| acetate copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 41 | 42 | 43 | 44 | 45 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 41a | 42a | 43 a | 44a | 45a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 41b | 42b | 43b | 44b | 45b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 46 | 47 | 48 | 49 | 50 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 46a | 47a | 48a | 49a | 50a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 46b | 47b | 48b | 49b | 50b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XE-30 | 0.1 to 10 | 0.15 to 5 | 0.15 to 5 | 0.2 to 2.5 | 0.2 to 2.5 |
| (specification as | | | | | |
| solid content) | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 51 | 52 | 53 | 54 | 55 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 51a | 52a | 53a | 54a | 55a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 51b | 52b | 53b | 54b | 55b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Polyvinylpyrrolidone | 1 to 10 | 2 to 8.5 | 2 to 8.5 | 3 to 7 | 3 to 7 |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 56 | 57 | 58 | 59 | 60 |
| Polymer a)* | 0.1 to 10 | 0.15 to 5 | 0.15 to 5 | 0.2 to 2.5 | 0.2 to 2.5 |
| Polyimide b) | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 56a | 57a | 58a | 59a | 60a |
| Guar | 0.1 to 10 | 0.15 to 5 | 0.15 to 5 | 0.2 to 2.5 | 0.2 to 2.5 |
| Hydroxypropyltrimonium | | | | | |
| Chloride** | | | | | |
| Polyimides-1 | 0.1 to 10 | 0.15 to 5 | 0.15 to 5 | 0.2 to 2.5 | 0.2 to 2.5 |
| Vinylpyrrolidone/vinyl | 1 to 10 | 2 to 8.5 | 2 to 8.5 | 3 to 7 | 3 to 7 |
| acetate copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 56b | 57b | 58b | 59b | 60b |
| N-Hance CCG 45 | 0.1 to 10 | 0.15 to 5 | 0.15 to 5 | 0.2 to 2.5 | 0.2 to 2.5 |
| (specification as | | | | | |
| solid content) | | | | | |
| Aquaflex XL-30 | 0.1 to 10 | 0.15 to 5 | 0.15 to 5 | 0.2 to 2.5 | 0.2 to 2.5 |
| (specification as | | | | | |
| solid content) | | | | | |
| Vinylpyrrolidone/vinyl | 1 to 10 | 2 to 8.5 | 2 to 8.5 | 3 to 7 | 3 to 7 |
| acetate copolymer | | | | | |
| PEG-40 Hydrogenated | 0.05 to 1.5 | 0.1 to 1 | 0.2 to 0.8 | 0.3 to 0.8 | 0.3 to 0.6 |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

*cationically modified guar derivative having a weight-average molecular weight of from about 5,000 to about 200,000 and a cationic degree of substitution (DS) of from about 0.1 to about 2.
**having a weight-average molecular weight of from about 5,000 to about 200,000 and a cationic degree of substitution (DS) of from about 0.1 to about 2.

“Misc” is to be understood to mean a cosmetic carrier, in particular (unless stated separately) water and possibly other customary constituents of styling products.

The cosmetic composition of the present disclosure can be formulated in the forms customary for the temporary reshaping of hair, for example, as a hair gel, hair spray, hair foam, hair cream or hair wax. Preference is given to the preparation as a hair gel.

Both hair foams and hair sprays require the presence of propellants. However, preferably no or only small amounts of hydrocarbons should be used for this purpose. Propane, propane/butane mixtures and dimethyl ether are particularly suitable propellants as contemplated herein.

The present disclosure also relates to the use of cosmetic compositions as contemplated herein for the temporary reshaping of keratinous fibers, in particular of human hair, and a method for the temporary reshaping of keratinous fibers, in particular human hair, in which the keratinous fibers are acted upon using a cosmetic composition as contemplated herein and are temporarily fixed in their shape.

With regard to further preferred embodiments of the use and the method, what has been said about the cosmetic compositions applies mutatis mutandis.

A further subject of this disclosure is the use of a cosmetic composition as contemplated herein for improving curl retention of temporarily reshaped keratinous fibers in a humid environment (HHCR).

### EXAMPLES

I. The following hair gels were prepared:

| Component/raw | INCI name or chemical | | | |
|---|---|---|---|---|
| material | name | V1 | V2 | E1 |
| N-Hance CCG 45 ¹ | Guar Hydroxypro- | 1 | — | 0.5 |
| | pyltrimonium | | | |
| | Chloride | | | |
| Aquaflex XL-30 ² | Polyimides-1 | — | 3.34 | 1.67 |
| Ethanol | | — | 96.66 | 48.33 |
| Water | | 99 | — | 49.5 |
| Total | | 100 | 100 | 100 |

¹ 92% by weight of active substance in water
² 28-32% by weight of active substance in water

The amount specifications in the table are given in % by weight of the respective raw material, based on the total composition.

For the styling agents obtained, the curl retention in a humid environment was determined by employing an HHCR test (High Humidity Curl Retention-Test: 6 h) on cleaned Kerling hair strands:

| | V1 | V2 | E1 |
|---|---|---|---|
| HHCR | 19.4% | 33.2% | 40.3% |

The polymer combination E1 as contemplated herein therefore showed a clearly super-additive, synergistic effect with respect to the degree of curl retention in a humid environment.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for the temporary reshaping keratinous fibers, comprising:
a) at least one cationically modified guar derivative having a weight-average molecular weight in the range of from about 5,000 to about 200,000 and a cationic degree of substitution in the range of from about 0.1 to about 2 and
b) at least one polyimide.

2. The cosmetic composition according to claim 1, wherein the weight proportion of the cationically modified guar derivative a) is from about 0.1 to about 10% by weight, based on a total weight of the cosmetic composition.

3. The cosmetic composition according to claim 1, wherein the cationically modified guar derivative a) is selected from the group of compounds having the INCI name Guar Hydroxypropyltrimonium Chloride.

4. The cosmetic composition according to claim 1, wherein the weight proportion of the polyimide b) is from about 0.1 to about 10% by weight, based on a total weight of the cosmetic composition.

5. The cosmetic composition according to claim 1, wherein the polyimide b) has at least one structural unit (A1), at least one structural unit (A2), at least one structural unit (A3) and at least one structural unit (A4), 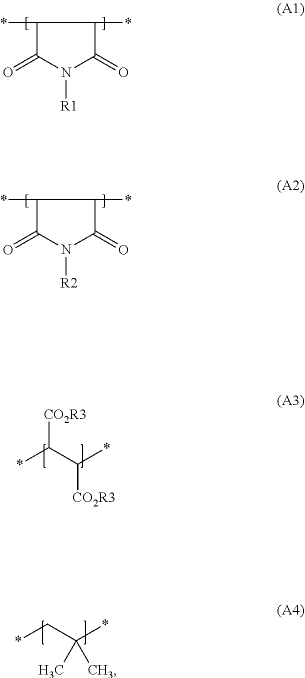
wherein
R1 stands for an optionally heterofunctionalized alkyl radical;
R2 stands for an optionally heterofunctionalized alkyl radical other than R1;
and
R3, independently of one another, stands for an optionally heterofunctionalized alkyl radical other than R1 and R2.

6. The cosmetic composition according to claim 1, wherein the polyimide b) is selected from the group of compounds having the INCI name isobutylene/dimethylaminopropyl maleimide/ethoxylated maleimide/maleic acid copolymer (Polyimide-1).

7. The cosmetic composition according to claim 1, wherein the cosmetic composition further comprises an alkanolamine or its neutralized form.

8. The cosmetic composition according to claim 1, wherein the composition further comprises an organic acid or its salt.

9. A method of using a cosmetic composition, the method comprising the steps of:
temporarily fixing keratinic fibers into a shape; and
applying the cosmetic composition to the keratinic fibers, wherein the cosmetic composition comprises a cationically modified guar derivative a) having a weight-average molecular weight in the range of from about 5,000 to about 200,000 and a cationic degree of substitution in the range of from about 0.1 to about 2 and a polyimide b).

10. The method of claim 9, wherein applying the cosmetic composition comprises applying the cosmetic composition wherein the modified guar derivative a) has the INCI name Guar Hydroxypropyltrimonium Chloride.

11. The method of claim 10, wherein applying the cosmetic composition comprises applying the cosmetic composition wherein the polyimide b) has the INCI name isobutylene/dimethylaminopropyl maleimide/ethoxylated maleimide/maleic acid copolymer (Polyimide-1).

12. The cosmetic composition of claim 1, wherein the modified guar derivative a) is present in the cosmetic composition at from about 0.2 to about 2.5 weight percent, based on a total weight of the cosmetic composition.

13. The cosmetic composition of claim 12, wherein the polyimide b) is present in the cosmetic composition at from about 0.2 to about 2.5 weight percent, based on the total weight of the cosmetic composition.

14. The cosmetic composition of claim 1 further comprising:
a polyvinylpyrrolidone.

15. The cosmetic composition of claim 14, wherein:
the polyvinylpyrrolidone is present in the cosmetic composition at from about 2 to about 8.5 weight percent, based on a total weight of the cosmetic composition.

16. The cosmetic composition of claim 1 further comprising:
the vinylpyrrolidone/vinyl acetate copolymer.

17. The cosmetic composition of claim 1, wherein:
a weight ratio of the cationically modified guar derivative a) to the polyimide b) is from about 5:1 to about 1:5.

18. The cosmetic composition of claim 17, wherein:
the weight ratio of the cationically modified guar derivative a) to the polyimide b) is from about 3:1 to about 1:3.

19. The cosmetic composition of claim 17, wherein:
the weight ratio of the cationically modified guar derivative a) to the polyimide b) is from about 1:1.1 to about 1.1:1.

20. The cosmetic composition of claim 1, wherein:
the modified guar derivative a) has the INCI name Guar Hydroxypropyltrimonium Chloride, and the modified guar derivative a) is present in the cosmetic composition at from about 0.2 to about 2.5 weight percent, based on a total weight of the cosmetic composition; and
the polyimide b) has the INCI name isobutylene/dimethylaminopropyl maleimide/ethoxylated maleimide/maleic acid copolymer (Polyimide-1) and is present in the cosmetic composition at from about 0.2 to about 2.5 weight percent, based on the total weight of the cosmetic composition
